(19) United States
(12) Patent Application Publication
(10) Pub. No.: US 2019/0038909 A1
(43) Pub. Date: Feb. 7, 2019

(54) A Therapeutic Method and Device Therefor

(71) Applicant: Helium 3 Resources Pty Ltd, Queensland (AU)

(72) Inventor: Raymond Dennis Palmer, Morley (AU)

(21) Appl. No.: 16/077,037

(22) PCT Filed: Feb. 10, 2017
(86) PCT No.: PCT/AU2017/050110

§ 371 Date: Aug. 9, 2018

(30) Foreign Application Priority Data
Feb. 10, 2016 (AU) 2016900450

(51) Int. Cl. A61N 5/06

(52) U.S. Cl. CPC A61N 5/062; A61N 5/0601; A61N 2005/067; A61N 2005/063; A61N 2005/0651; A61N 2005/0602

(57) ABSTRACT

Methods and devices for delivering electromagnetic radiation for therapeutic purposes are provided herein. The methods and devices include contacting blood with electromagnetic radiation, preferably by an optical fibre. The methods and devices are particularly suitable for intra-arterial irradiation of a treatment site of a subject.

### TECHNICAL FIELD

THIS disclosure relates to therapeutic and/or delivery methods and systems. More particularly, the present disclosure relates to methods for delivery of electromagnetic radiation for the treatment of diseases, disorders or conditions responsive to electromagnetic radiation.

### RELATED APPLICATION

This application claims the benefit of Australian Provisional Application No. 2016900450, filed on 10 Feb. 2016, the entire contents of which is hereby incorporated by reference herein.

### BACKGROUND

The burden on healthcare systems due to surgical procedures on difficult to access areas of the body is considerable. Treatment methods for such problematic areas that are cost-effective, relatively non-invasive and targeted are attractive to not only patients but also clinicians and health care administrators. By way of example, photodynamic therapy (PDT) is a combination treatment for cancer that utilises light to activate photosensitising agents that have been absorbed by the cancer cells. Although an attractive treatment modality due to its lack of adverse events, non-invasive nature, can be targeted to the site of action and speed, PDT is only useful to treat areas where light can reach such as skin and the endothelial lining that can be reached with a light source (e.g., mouth). Treatment of deep, internal sites by light is difficult due to the short transmission path of light.

It will be appreciated that reference herein to “preferred” or “preferably” is intended as exemplary only.

### SUMMARY

The present disclosure is predicated, in part, by the recognition that blood serves as an excellent optical transmission medium for the targeted delivery of electromagnetic radiation, and in particular light, to inaccessible or remote body sites that require treatment with curative or therapeutic doses of electromagnetic radiation. In some broad aspects, the present disclosure relates to methods and devices for introducing and transmitting electromagnetic radiation through blood in a non-linear path. In some examples described below, aspects of the invention will be described in relation to light, though the invention is not so limited.

In a first aspect, there is provided a method of delivering electromagnetic radiation to a subject, including the step of contacting blood, a cell, a surface, a tissue and/or an organ of the subject with an electromagnetic radiation such that the electromagnetic radiation is transmitted through blood to a site in need thereof, or is transmitted to the cell, the surface, the tissue and/or the organ of the subject, to thereby deliver electromagnetic radiation to the subject.

In a second aspect, there is provided a method of preventing and/or treating a disease, disorder and/or condition that is responsive to treatment with an electromagnetic radiation in a subject, said method including the step of administering a therapeutically effective amount of the electromagnetic radiation via a vessel of the blood circulatory system to a site in need thereof, to thereby treat said disease, disorder and/or condition in said subject.

Preferably, the electromagnetic radiation is delivered or administered by at least one optical fibre capable of transmitting electromagnetic radiation, and more preferably capable of transmitting electromagnetic radiation through blood.

In a third aspect, there is provided a device for targeted delivery of electromagnetic radiation to a subject, said device comprising:

a first optical fibre capable of transmitting electromagnetic radiation through blood to a site in need thereof or is capable of transmitting electromagnetic radiation to a cell, a surface, a tissue and/or an organ of the subject; and

optionally, a second optical fibre capable of receiving light.

Preferably, targeted delivery is to a cell, a surface, a tissue and/or an organ of said subject.

Suitably, the second optical fibre is a camera.

In other preferred embodiments, the first optical fibre is capable of transmitting electromagnetic radiation and is capable of receiving light. In these preferred embodiments, the first optical fibre has a dual function as a camera and a transmitter of electromagnetic radiation and more preferably, light.

Preferred embodiments of the third aspect provide for targeted delivery of light to a subject.

In a fourth aspect, there is provided a method delivering electromagnetic radiation to a subject using the device of the third aspect.

The device may include a securing member configured to secure the device to at least a portion of the subject. The securing member may preferably include includes strapping.

In a fifth aspect, there is provided a method of preventing and/or treating a disease, disorder and/or condition that is responsive to treatment with an electromagnetic radiation in a subject using the device of the third aspect.

In a sixth aspect, there is provided a method for activating a photosensitive drug in a remote location in a subject, the method including: delivering the photosensitive drug to a target area in need of treatment; moving a fibre optic light source into contact with a blood stream in fluid communication with the target treatment area; and delivering light to the target treatment area to activate the photosensitive drug, the delivering of the light including transmitting the light from the fibre optic source into the blood stream along a non-linear path to the target treatment area.

The method of the sixth aspect may include a fibre optic light source configured to transmit non-visible light.

The methods of the first, second or sixth aspect may further comprise injecting a blood-thinning agent or drug into the blood stream prior to delivering the light to the target treatment area.

In a seventh aspect, there is provided a system for intra-arterially irradiating a target treatment area within a subject, comprising: an electromagnetic radiation source including at least one optic fibre configured for passage through a catheter, said optic fibre including a tip adapted to transmit electromagnetic radiation to a blood stream of the subject; an adjustment means for adjusting a frequency or wavelength of the electromagnetic radiation; and a processor configured to adjust the frequency of the electromagnetic radiation based on an estimated or measured distance from said tip to the target treatment area.

Preferably, the subject according to any one of the aforementioned aspects is a mammal and more preferably, the mammal is a human. The present disclosure also contemplates treatment of mammals other than humans inclusive of domestic livestock and companion animals.

Suitably, the electromagnetic radiation is within the range of wavelengths between and inclusive of ultraviolet and infrared. Preferably, the electromagnetic radiation is light, more preferably infrared light and visible light. Even more preferably, the light is visible light.

In preferred embodiments of any one of the aforementioned aspects, the electromagnetic radiation source is selected from the group consisting of a laser, a light emitting diode, an electroluminescent panel, an arc lamp, an incandescent light source, a light emitting device and any combinations thereof. In preferred embodiments, the electromagnetic radiation source is a light emitting diode and more preferably a plurality of light emitting diode. In preferred embodiments, the light emitting diode is an organic light emitting diode or a quantum dot light emitting diode. In other preferred embodiments, the electromagnetic radiation source is a laser. In yet other preferred embodiments, the electromagnetic radiation source is an electroluminescent panel. Other preferred embodiments relate to a plurality of electromagnetic radiation sources.

In preferred embodiments of any one of the aforementioned aspects, the electromagnetic radiation source is configured to emit a wavelength or wavelengths, or the electromagnetic radiation source has a wavelength, between about 10 nanometres and about 1 millimetre, more preferably between about 100 nanometres and about 500 micrometres, even more preferably 400 nanometres and about 100 micrometres and yet even more preferably between about 400 nanometres and about 10 micrometres. In further preferred embodiments, the electromagnetic radiation source is configured to emit a wavelength between about 10 nanometres and about 1200 nanometres, more preferably about 400 nanometres and about 1000 nanometres, even more preferably between about 630 nanometres and about 790 nanometres and yet even more preferably, a wavelength selected from the group consisting of about 630 nanometres, about 653 nanometres, about 660 nanometres and about 750 nanometres, and any combination thereof. In certain preferred embodiments of any one of the aforementioned aspects, the electromagnetic radiation source is configured to emit a wavelength between about 800 nanometres to about 1200 nanometres, and more preferably, about 830 nanometres. In other preferred embodiments, the electromagnetic radiation source is configured to emit a plurality of wavelengths.

Unless the meaning is clearly to the contrary, all ranges set forth herein are deemed inclusive of the endpoints. Ranges are to be interpreted as being fully inclusive of all values between the limits.

The articles “a” and “an” are used herein to refer to one or to more than one (i.e., to at least one) of the grammatical object of the article. By way of example, “an element” means one element or more than one element. As used herein, the use of the singular includes the plural (and vice versa) unless specifically stated otherwise.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The claims as filed and attached with this specification are hereby incorporated by reference into the text of the present description.

### BRIEF DESCRIPTION OF THE DRAWINGS

In order that the invention may be readily understood and put into practical effect, reference will now be made to embodiments of the invention with reference to the accompanying drawings, wherein like reference numbers refer to identical elements. The drawings are provided by way of example only, wherein:

FIG. 1 is a diagrammatic representation of optic fibre tip arrangements in accordance with exemplary embodiments of the present disclosure.

FIG. 2 is a diagrammatic representation of a device in clinical practice in accordance with an exemplary embodiment of the present disclosure.

FIG. 3 is a further diagrammatic representation of the device in clinical practice as shown in FIG. 2.

FIG. 4 is a yet further diagrammatic representation of the device in clinical practice as shown in FIG. 2.

Skilled addressees will appreciate that elements in the drawings are illustrated for simplicity and clarity and have not necessarily been drawn to scale. By way of example, the relative dimensions of some of the elements in the drawings may be distorted to help improve understanding of embodiments of the invention.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

Each embodiment described herein is to be applied mutatis mutandis to each and every embodiment unless specifically stated otherwise.

The methods and devices of the present disclosure have arisen, at least in part, from the recognition that blood can be used as an optical transmission medium for the specific and targeted delivery of electromagnetic radiation, and more particularly light, to a site in need of therapy. A particular (and non-limiting) advantage conferred by embodiments of the methods and devices of the present disclosure is the ability to access internal organs, surfaces, tissues and/or cells that are not conveniently located for, or readily accessible to, conventional electromagnetic radiation therapeutic techniques.

Known techniques and devices for delivery of electromagnetic radiation are hampered due, in part, to an inability of the electromagnetic energy to transmit efficiently through blood to a site requiring therapy. In an exemplary aspect, the present disclosure utilises an alternative light delivery mechanism to standard optical fibres. Preferably, the light emitted from a tip of an optical fibre in accordance with the present disclosure is wide angle such that the light is not focused into a pin point, but travels out in a broader radius to achieve maximum light transmission through the blood. Accordingly, as the light leaves the tip of a first optical fibre and enters into the blood, the light is carried by the water in the blood around curves and bend of the arteries and into capillaries to deliver electromagnetic radiation (usually in the form of light) to the target area. It will be appreciated that in this way, the patient's own blood is used to supply to carry and deliver the electromagnetic radiation and thus acts as a “biological optical fibre” transmitting medium. Suitably and in accordance with the present disclosure, the blood permits the transmission of the electromagnetic radiation in a non-linear path through the vessels to the site of action, as is required. In particular embodiments, it may be necessary to inject a patient with blood thinning liquids and additional fluids to maximize the optical transmittance of the blood. In alternative embodiments, an optical fibre transmits light as a substantially focused beam pathway and according to preferred forms of these embodiments, electromagnetic radiation is transmitted to the site in need thereof without transmission through blood. In exemplary embodiments, a blood thinning agent may be water, or a pharmaceutic such as warfarin, although without limitation thereto.

Preferred embodiments relate to a method of delivering electromagnetic radiation to a subject, including the step of contacting blood with an electromagnetic radiation such that the electromagnetic radiation is transmitted through blood to a site in need thereof, to thereby deliver electromagnetic radiation to the subject. Alternative preferred embodiments relate to a method of delivering electromagnetic radiation to a subject, including the step of contacting a cell, a surface, a tissue and/or an organ of the subject with an electromagnetic radiation such that the electromagnetic radiation is transmitted to the cell, the surface, the tissue and/or the organ of the subject, to thereby deliver electromagnetic radiation to the subject.

In preferred embodiments, the step of contacting occurs in vivo and thereby occurs in a subject. In other suitable embodiments, the step of contacting is ex vivo and the treated material is delivered or administered to the subject. Suitably, the step of contacting blood may be by way of intra-arterial or intra-venous delivery.

In other embodiments, the present disclosure relates to devices for targeted delivery of electromagnetic radiation to a subject, said device comprising: a first optical fibre capable of transmitting electromagnetic radiation through blood to a site in need thereof; and optionally, a second optical fibre capable of receiving light. In yet other preferred embodiment, the present disclosure relates to devices for targeted delivery of electromagnetic radiation to a subject, said device comprising: a first optical fibre capable of transmitting electromagnetic radiation to a cell, a surface, a tissue and/or an organ of the subject; and optionally, a second optical fibre capable of receiving light.

In preferred embodiments, targeted delivery is to a cell, a surface, a tissue and/or an organ of the subject. The electromagnetic radiation at least partially penetrates the desired target. The level of penetration may be dependent on what is being treated, or the disease, disorder and/or condition to be treated.

In preferred embodiments of the methods of the present disclosure, a step of administering, delivering or contacting with an electromagnetic radiation is remote to the site in need thereof, or the target area.

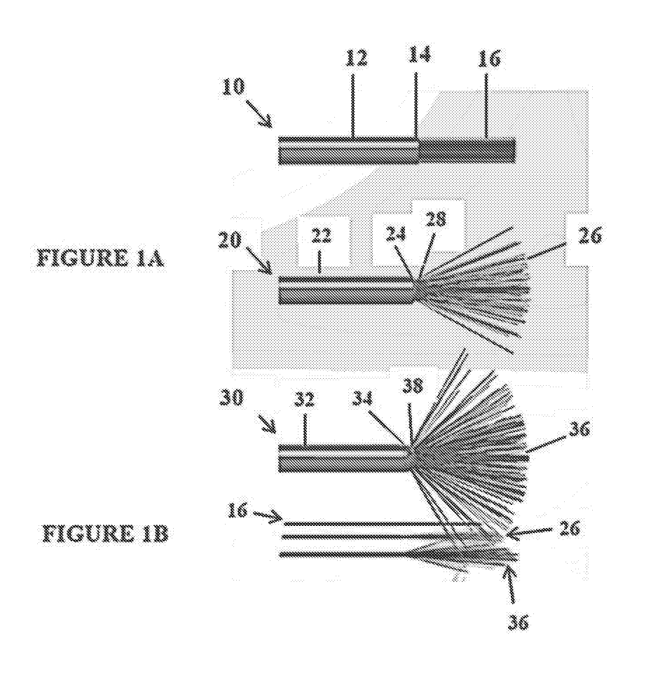

Reference is made to FIG. 1A and FIG. 1B, which depicts non-limiting configurations of one or more suitable beam pathways emitted from a tip of an optical fibre in accordance with the present disclosure. The present disclosure contemplates a tip of the optic fibre with or without a diffuser to create alternative beam pathways. In FIG. 1A, reference numerals 10, 20 and 30 generally indicates exemplary embodiments of an optical fibre. An optical fibre 10 includes a sheath 12, a tip 14 and a beam pathway 16. Sheath 12 may house the electrical components for the optical fibre. Sheath 12 may house or direct a beam travelling through optical fibre 10. Sheath 12 may be formed from a plastics material, or a metal material, although without limitation thereto. Beam pathway 16 is emitted from tip 14 in a substantially straight, linear conformation.

An optical fibre 20 includes a sheath 22, a tip 24, a beam pathway 26 and a diffuser 28. Diffuser 28 is located at or near tip 24. Sheath 22 may house the electrical components for the optical fibre. Sheath 22 may house or direct a beam travelling through optical fibre 20. Sheath 22 may be formed from a plastics material, or a metal material, although without limitation thereto. Beam pathway 26 is emitted from tip 24 at various angles, and more preferably a semi-wide angle beam. Diffuser 28 may be integral with, or formed with tip 24. Diffuser 28 may be removably attachable to tip 24. Diffuser 28 is sized and configured to diffuse or disperse a flow of light travelling therethrough. Accordingly, diffuser 28 preferably includes one or more apertures (not shown) which serve to diffuse or disperse a flow of light travelling therethrough. The, or each, aperture may be of varying sizes and/or shapes in order to achieve a beam pathway having different angles and characteristics that are suitable for a desired or particular purpose in accordance with the present disclosure. It will be appreciated that diffuser 28 may include arrangements other than one or more apertures to diffuse or disperse the flow of light travelling therethrough.

An optical fibre 30 includes a sheath 32, a tip 34, a beam pathway 36 and a diffuser 38. Diffuser 28 is located at or near tip 24. Sheath 22 may house the electrical components for the optical fibre. Sheath 32 may house or direct a beam travelling through optical fibre 30. Sheath 32 may be formed from a plastics material, or a metal material, although without limitation thereto. Beam pathway 36 is emitted from tip 34 at various angles, and more preferably a wide angle beam pathway. Diffuser 38 may be integral with, or formed with tip 34. Diffuser 38 may be removably attachable to tip 34. Diffuser 38 is sized and configured to diffuse or disperse a flow of light travelling therethrough. Accordingly, diffuser 38 preferably includes one or more apertures (not shown) which serve to diffuse or disperse a flow of light travelling therethrough. The, or each, aperture may be of varying sizes and/or shapes in order to achieve a beam pathway having different angles and characteristics that are suitable for a desired or particular purpose in accordance with the present disclosure. It will be appreciated that diffuser 38 may include arrangements other than one or more apertures to diffuse or disperse the flow of light travelling therethrough.

In FIG. 1B, there is shown beam pathway 16, 26 and 36 of optical fibres 10, 20 and 30 without the accompanying sheath 12, 22 and 32 respectively.

In preferred embodiments, one or more angles of a beam pathway may be in a range from about +170° to about −170°, about +160° to about −160°, about +150° to about −150°, about +140° to about −140°, about +130° to about −130°, about +120° to about −120°, about +110° to about −110°, about +100° to about −100°, about +90° to about −90°, about +80° to about −80°, about +70° to about −70°, about +60° to about −60°, about +50° to about −50°, about +45° to about −45°, about +40° to about −40°, about +35° to about −35°, about +30° to about −30°, about +25° to about −25°, about +20° to about −20°, about +15° to about −15°, about +10° to about −10°, about +5° to about −5°, about +2° to about −2°, about +1° to about −1° and about +0.5° to about −0.5°, and all values between these limits. It will be appreciated that the recited angles are relative to a “zero” angle beam, being a substantially straight beam emitted from a tip without diffusion or dispersion at an angle. An angle as described herein may be referred to as a “detection angle”, as is known in the art.

The beam pathway may include any geometrical dispersion and/or intensity that is suitable for a particular purpose. The geometrical dispersion or intensity may be changed in accordance with the particular application according to the present disclosure, such as, but not limited to, the treatment modality, disease to be treated and duration of treatment.

All frequencies of electromagnetic radiation are contemplated for use in the methods and devices as described herein and preferably ultra-violet, visible and/or infrared. It will be appreciated that the choice of wavelength may be dependent, at least in part, on the depth of the targeted treatment area. Generally, although not exclusively, shorter wavelengths of less than or equal to about 700 nanometres (nm) are used to treat superficial tissue whereas wavelengths above about 700 nm penetrate further and are typically used to treat deeper-seated tissue. It will be appreciated that typically, although not exclusively, Blue light of about 400 nm to about 470 nm has depth of light penetration of <1 mm; Blue—green light between about 475 nm to about 545 nm has depth of 0.3 to 0.5 mm; Yellow light of about 570 nm to about 590 nm has a depth of 0.5 mm to 2 mm; red light of about 630 nm to about 790 nm has depth of 2 to 3 mm and near infra-red of about 800 nm to about 1200 nm has a depth of 5 mm to 10 mm.

The present disclosure also contemplates wavelengths in the mid infra-red and far infra-red ranges (collectively spanning a range between about 1200 nm and up to about 1 millimetre) of the electromagnetic spectrum. In general embodiments, the wavelength is preferably in the range between about 10 nm to about 1 millimetre, more preferably between about 100 nanometres and about 500 micrometres, even more preferably between about 200 nanometres and about 300 micrometres, yet even more preferably 400 nanometres and about 100 micrometres and yet even more preferably between about 400 nanometres and about 10 micrometres. In certain preferred embodiments, the electromagnetic radiation source is configured to emit a wavelength between about 400 nanometres and about 1 millimetre and more preferably, between about 800 nm and about 1 millimetre. In further general preferred embodiments, the wavelength may be in the range between about 10 nm to about 1200 nm. Preferably, the electromagnetic radiation is light in the visible range of the electromagnetic spectrum. In preferred embodiments, the wavelength of the electromagnetic radiation source is between about 400 nm and about 1000 nm, more preferably between about 400 nm and about 700 nm, even more preferably about 630 nm to about 1200 nm, even more preferably about 630 nm to about 790 nm. In other preferred embodiments, the wavelength is in a range between about 800 nm to about 1200 nm. The cited ranges include about 20 nm, about 50 nm, about 100 nm, about 200 nm, about 300 nm, about 400 nm, about 500 nm, about 600 nm, about 700 nm, about 800 nm, about 900 nm, about 1 micrometre, about 1.2 micrometres, about 10 micrometres, about 50 micrometres, about 100 micrometres, about 200 micrometres, about 300 micrometres, about 400 micrometres, about 500 micrometres, about 600 micrometres, about 700 micrometres, about 800 micrometres, about 900 micrometres and about 1 millimetre. In advantageous preferred embodiments, a wavelength is selected from the group consisting of about 630 nanometres, about 653 nanometres, about 660 nanometres, about 750 nanometres and about 830 nanometres, and any combination thereof. These ranges are to be interpreted as being fully inclusive of all values between the limits. These ranges are inclusive of the endpoints.

The present disclosure contemplates methods and devices to irradiate a target area that uses a single wavelength (monotherapy) as well irradiating a target area with a combination of different wavelengths (so called ‘combination treatment’). Combination treatment may be useful to target various depths of tissue. Although the wavelengths are different in a combination treatment modality, other parameters such as dose, fluence, power density, pulse structure and timing may be the same or different. Such parameters will depend on the application, as will be known by a person of skill in the art. By way of example, a combination treatment may comprise a wavelength from the red spectra and a wavelength from the near infra-red at the same or different fluence, dose, power density, pulse structure and timing. A treatment regime may include a single exposure or alternatively, a treatment course of over a number of hours, days or weeks, as is required. In certain preferred embodiments, a wavelength in the near infrared range may be particular suitable for treatment of multiple sclerosis. A particularly useful wavelength for treatment of multiple sclerosis is in the near infrared range, and preferably 670 nm. In one preferred aspect, a method of the present disclosure utilises one or more photosensitizing drugs. The photosensitizing drug is delivered to the target area (e.g., an area proximate a tumour). An optic fibre is moved through a catheter needle to protrude out from the centre of the needle into a blood vessel (e.g., artery). The optic fibre is fed through the artery towards the target area. A non-harmful light intensity is transmitted by the optic fibre into the blood from the tip of the optic fibre and travels through the artery to the location of the photosensitizing drug. A non-limiting advantage of this delivery treatment system and method is the ability to reach remote, conventionally inaccessible areas to activate photosensitive drugs with an intra-arterial and/or intravenous light source. Another advantage of the exemplary system and method is that it permits a more targeted and effective treatment in a less invasive fashion compared to conventional treatments. Non-limiting examples of suitable photosensitizing drugs may include porfimer sodium (e.g., Photofrin®), aminolevulinic acid (ALA; e.g., Levulan®) and a methyl ester of ALA (e.g., Metvixia® cream).

In another preferred aspect, a dual core optic fibre is utilised. One optic fibre transmits light, while a second optic fibre receives light. The second optic fibre may be utilised as a camera. An advantage of the dual core configuration is that it permits more precise targeting of light delivery to the target treatment area. One or both optic fibres may have a length in the range of 100 mm to 600 mm. The dual core configuration may be used to move the light source to an area proximate the target treatment area, with the light being delivered over final distance to the target treatment area via the blood stream, in a similar fashion to that described above. It will be appreciated that light can be delivered to the target treatment area without the dual core configuration if desired. It will be further appreciated that a dual configuration permits treats treatment and viewing with an optical single fibre. The dual configuration in some preferred embodiments is a single optical fibre that emits light or other electromagnetic radiation and is configured as a camera.

The present disclosure has particular utility for the prophylactic or therapeutic treatment of diseases, disorders and/or conditions that are responsive to electromagnetic radiation therapy and in particular, light therapy. In preferred embodiments, the present disclosure is suited to delivery of electromagnetic radiation through the blood circulatory system to a site in need thereof. The vessels of the blood circulatory system comprise veins, arteries and capillaries. Preferably, electromagnetic radiation is delivered via an artery and/or a capillary. It is envisaged that this mode of delivery permits access to difficult to access sites in the body.

It will be appreciated that the methods and devices of the present disclosure are amenable to use on any disease, disorder and/or condition (on any part of the human body) that is sensitive to electromagnetic radiation and more preferably, light.

The present disclosure is particularly suited for use in photodynamic therapy for the treatment of cancerous growths, solid tumours and metastatic cancers, breast cancer, pancreatic cancer, brain cancer, although without limitation thereto. Agostinis et al. (2011, CA Cancer J Clin 61(4):250-281) provides an informative review of photodynamic therapy of cancer and is incorporated herein by reference.

It will be appreciated that the present disclosure may relate to therapeutic treatment of a cancer in the absence of photodynamic therapy. Near Infrared Radiation (NIR) has been demonstrated to have an inhibitory effect on advanced neoplasia (cancer). Broad-spectrum irradiation ranging between 1100 nm and 1800 nm results in apoptosis (cell death) in multiple cancer cell types in vitro, independent of thermal energy. The cancer may be a breast cancer. According to embodiments that contemplate a breast cancer, an exemplary wavelength range is 480 nm to 3400 nm. The cancer may be a pancreatic cancer. According to embodiments that contemplate a pancreatic cancer, an exemplary wavelength range is between 1100 nm and 1800 nm. An exemplary method that include PDT may include 480-3400 nm, 95% polarization, 40 mW/cm² and 24 J/cm². This may be particularly useful for treatment of a breast cancer, and in particular post-surgical breast cancer patients.

The present disclosure is also amenable for treatment of neurological disorders, diseases and/or conditions potentially responsive to electromagnetic radiation therapy such as Alzheimer's disease, dementia, Multiple Sclerosis, Parkinson's Disease, a mild traumatic brain injury, stroke and other cognitive and emotional conditions. In those embodiments that relate to Alzheimer's disease, a particularly suitable spectrum is in the red to infrared spectrum, and in particular and in particular light in the near infrared (NIr) range and particularly preferable between 600 nm and 1070 nm. An exemplary method for treatment of a mild brain injury may include a fluence rate of 1 J/cm²·min for a power density of 16.67 mW/cm², with a peak spectral wavelength at steady-state temperature (42.2° C.) of about 903 nm.

Other types of disease, disorders and/or conditions that may benefit from the methods and devices of the present disclosure include depression (by chemical rebalance), internal organs, and in particular the interior of such organs, that would benefit from treatment with electromagnetic radiation such as heart muscle, liver, kidneys, bladder, bowel and breasts, without limitation thereto. By way of example only, the methods and devices of the present disclosure are amenable to delivery of light into a heart chamber and past an active valve whilst it is beating to provide therapeutic benefit, as required.

The present disclosure has utility in nerve regeneration. Near-infrared light-emitting diodes may be applicable for nerve regeneration.

It will be appreciated that in reference to the present disclosure, blood acts as a transmission medium for delivery of therapeutically effectively amounts of electromagnetic radiation to a site in need thereof. That is, in the context of the present disclosure, blood can be thought of as a carrier medium, although without limitation thereto. Accordingly, in one preferred aspect, the present disclosure excludes the therapeutic treatment of a blood, in the form of, for example, in vivo blood irradiation (e.g., blood infections), phototherapy of blood, or any act that constitutes the therapeutic intervention or treatment of blood itself. In another preferred aspect, the blood itself can be treated by, for example, transmitting the light throughout the components of the blood circulatory system and in particular, the arterial system.

By “a site in need thereof” is meant a site that requires therapy or is targeted for therapy and can be a cell, a surface, a tissue and/or an organ, although without limitation thereto.

The term “therapeutically effective amount” describes a quantity of a specified agent sufficient to achieve a desired effect in a subject being treated with that agent. In some embodiments, a “therapeutically effective amount” is a sufficient amount of electromagnetic radiation to inhibit growth of a cancer. In other embodiments, “therapeutically effective amount” is a sufficient amount of electromagnetic radiation to regenerate nerve endings in a Multiple Sclerosis patient. In alternative embodiments, a “therapeutically effective amount” is an amount for use in photodynamic therapy. The “therapeutically effective amount” will vary depending upon the health and physical condition of the subject and the taxonomic group of individual to be treated, the formulation of the composition, the assessment of the medical situation, and other relevant factors.

Ideally, a “therapeutically effective amount” is an amount sufficient to induce the desired result without causing a substantial adverse effect (such as a cytotoxic effect or allergic effect, but without limitation thereto) in the subject. The therapeutically effective amount will vary depending upon the health and physical condition of the subject and the taxonomic group of individual to be treated, the formulation of the composition, the assessment of the medical situation, manner of administration, the severity of the disease, disorder and/or condition being treated, and other relevant factors. Administration of a therapeutically effective amount of an active agent to a subject, may either in a single dose or as part of a series (for example daily) or slow release system, during a course of treatment. However, the frequency of administration is dependent on the preparation applied, the subject being treated, the severity of disease, disorder and/or condition and the manner of administration of the therapy or composition.

By “administering” or “administration” is meant the introduction of a form of electromagnetic radiation into a subject by a particular, chosen route.

The terms “patient”, “subject”, “host” or “individual” used interchangeably herein, refer to any subject, particularly a vertebrate subject, and even more particularly a mammalian subject, for whom therapy or prophylaxis is desired. Suitable vertebrate animals that fall within the scope of the present disclosure include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice, rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars), marine mammals (e.g., dolphins, whales), reptiles (e.g., snakes, frogs, lizards), and fish. In specific embodiments, the subject is a primate such as a human. However, it will be understood that the aforementioned terms do not imply that symptoms are present.

As used herein, the terms “prevent,” “prevented,” or “preventing,” refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

As used herein, the terms “treatment,” “treating,” and the like, refer to obtaining a desired pharmacologic and/or physiologic effect. The effect may be therapeutic in terms of a partial or complete cure for a disease or condition (e.g., a hyperproliferative cell disorder such as cancer) and/or adverse effect attributable to the disease or condition.

These terms also cover any treatment of a condition or disease in a mammal, particularly in a human, and include: (a) inhibiting the disease or condition, i.e., arresting its development; or (b) relieving the disease or condition, i.e., causing regression of the disease or condition.

The present disclosure relates to systems for intra-arterially irradiating a target treatment area within a subject, comprising: an electromagnetic radiation source including at least one optic fibre configured for passage through a catheter, said optic fibre including a tip adapted to transmit electromagnetic radiation to a blood stream of the subject; an adjustment means for adjusting a frequency or wavelength of the electromagnetic radiation; and a processor configured to adjust the frequency of the electromagnetic radiation based on an estimated or measured distance from said tip to the target treatment area. The optic fibre may be a dual configuration optic fibre.

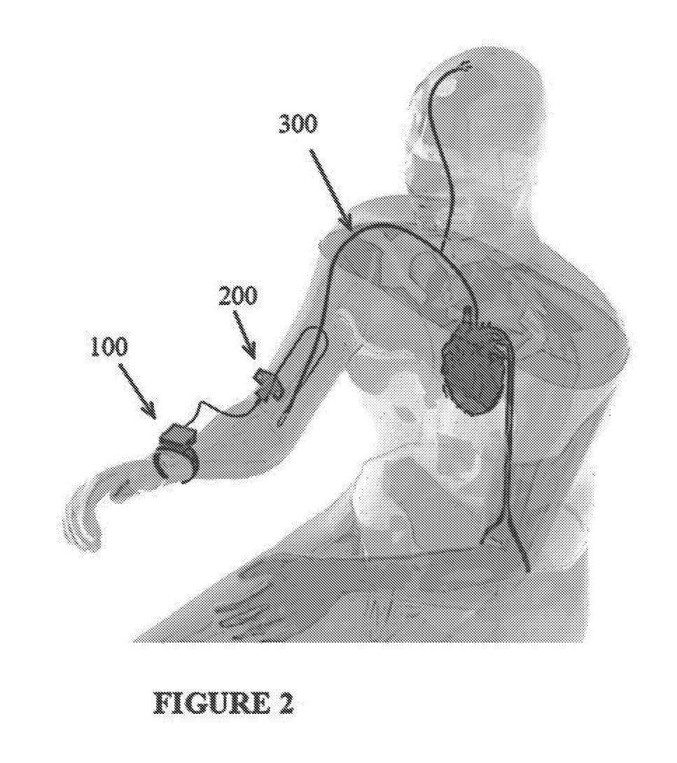

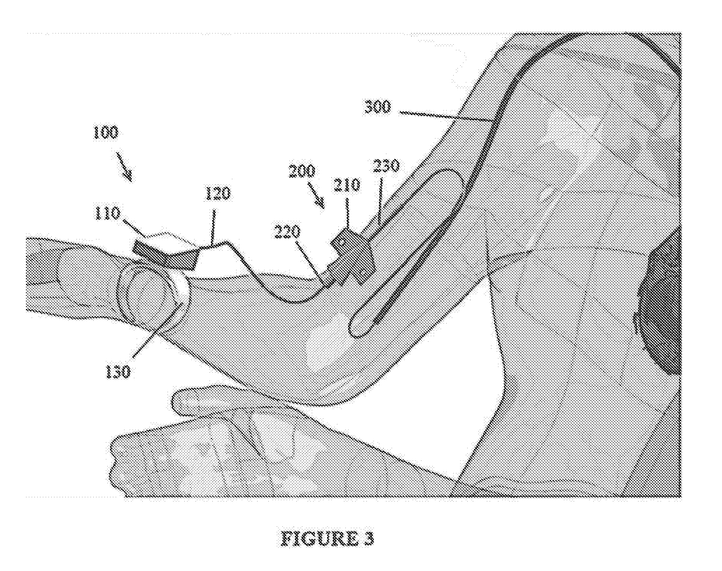

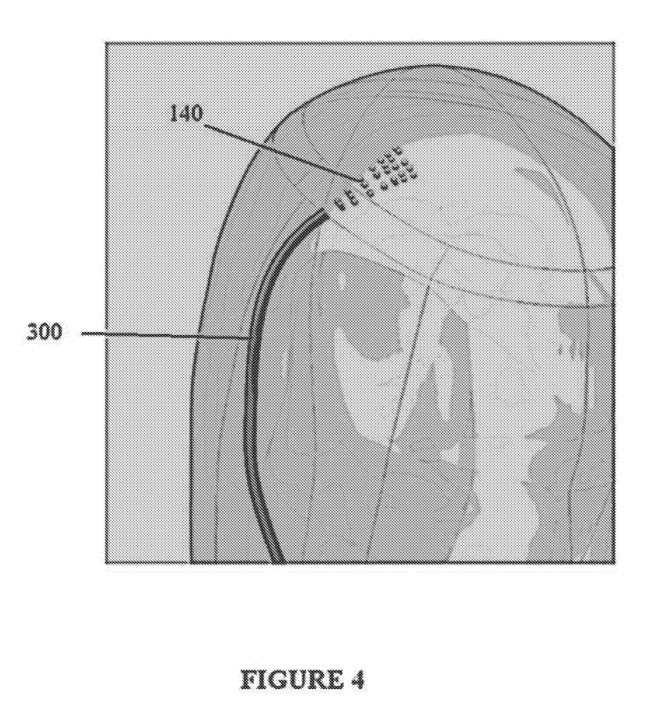

Reference is made to FIGS. 2 to 4, which show a device 100 of the present disclosure in clinical practice. Device 100 comprises a body 110 attached or engaged with a securing member 130 configured for securing device 100 to a subject, in this case a wrist. Securing member 130 may include strapping. Body 110 may include a source of electromagnetic radiation. It is also contemplated that body 110 may include a manual input interface for a user to input one or more parameters, or a display in order to provide feedback to a user. An optical fibre 120 is connected to body 110. In clinical practice, optical fibre 120 of device 100 may be thread through an aperture 220 of a catheter 230 of cannula 200 (positioned on a suitable surface for access to the target area such as a dermal surface, although without limitation thereto) and into an artery 300 to the targeted or diseased area, which in the depicted embodiment is the brain (although without limitation thereto). A beam pathway 140 is administered to the target therapeutic site. Optical fibre 120 may be of any suitable length to achieve this purpose. Cannula 200 is as known in the art and may include a butterfly body 210 to assist with stability. In addition, the optical fibres present in the devices or used in the methods of the present disclosure may be manufactured from any suitable material such as, but not limited to, glass and plastic. A particular advantage of those embodiments that contemplate inclusion of a camera in a device of the present disclosure is highly accurate targeting of electromagnetic radiation to the desired therapeutic site. Device 100 is present on the wrist of a subject but it is envisaged that device 100 may be present on any party of subject's body e.g., chest. It is also contemplated that device 100 may not be configured to attach to the subject's body but may be remote to the subject's body. It is contemplated that a method of detecting optical transmission is to insert a fibre optic through a cannula into a subject's arm, beaming light up into their artery, on the other arm and inserting another cannula with a receiving optical fibre attached to a spectroradiometer to demonstrate the transmission of light through blood and also to measure the amount of absorbance and energy being delivered.

According other embodiments, a catheter needle may be placed into a vein or artery close to the problem area. The thin fibre optic cable may then be fed into the catheter needle and pushed deep up into the vein or artery until the specialist is happy that the tip is close to the target area. The other end of the fibre optic cable, which is outside the body, is plugged into the specific light source which has been selected for the specific wavelength and treatment required. The non-harmful intense light is transmitted into the optic fibre, it travels through the fibre and out of the tip to the treatment area or carried further by the fluids in the blood to the target area. The light travels via basic optical light transmittance theory through the arterial system to where the photosensitizing drugs have taken effect on the cancer cells. Light is now penetrating at inoperable areas of the human anatomy, where cancer cells may reside. Even in the tiniest of capillaries and blood vessels, it is contemplated that the light delivery technique described herein will have utility, because where there is blood, there is water, where there is water, there is an optical transmission medium. In particular embodiments, delivered to the blood supply, a wavelength of light which will interact with the photosentizing drugs used for PDT to treat a tumour, which requires a blood supply to grow and exist. It is envisaged that this may be effective at killing the desired cells, e.g., metastasized cancer cells, that have made it into the blood stream and are metastasizing elsewhere. Once close enough, the light leaves the tip of the fibre and uses the blood to carry/transmit it further to the treatment area. The light transmittance ability of blood could easily be increased with simple fluids.

The electromagnetic radiation source contemplated for use in the present invention is configured to emit electromagnetic radiation at one or more desired wavelengths. Preferably, the electromagnetic radiation source is selected from the group consisting of a laser, a light emitting diode, an electroluminescent panel, an arc lamp and an incandescent light source, a light emitting device and any combinations thereof. In certain preferred embodiments, the electromagnetic radiation source uses photons at a non-thermal irradiance to alter a biological activity, otherwise termed ‘low level light therapy’. In other preferred embodiments, the electromagnetic radiation source is a coherent light source (laser) or a non-coherent light source such as a filtered lamp or a light emitting diode (LED), or a combination of a coherent light source and a non-coherent light source. In suitable embodiments that relate to low level light therapy, the electromagnetic radiation source is a coherent light source or a non-coherent light source, or a combination thereof. According to the embodiments relating to low level light therapy, the non-coherent light source is a filtered lamp or a light emitting diode, or a combination thereof.

In preferred embodiments, the electromagnetic radiation source includes at least one light emitting diode (LED), as is known in the art. The LED device may be configured to emit one or a plurality of wavelengths or spectral ranges. By way of example, an LED device may be a red LED that emits only in the red spectral range. Alternatively, the LED device may be configured to emit electromagnetic radiation in the red spectral range and the near infra-red spectral range. In some preferred embodiments, the LED is an organic LED (‘OLED’). An OLED, as would be known by a skilled addressee, emits light due to electroluminescence of thin films of organic semiconductors. An OLED comprises a light emitting organic material that is suitable for use in an OLED. Preferably, the light emitting organic material is selected from a small molecule, a polymer, a dendrimer and any combinations thereof. It will be appreciated that an OLED may have a plurality of layers comprising a light emitting organic material. In an OLED, the organic semiconductors may be deposited on a glass substrate or on substrate constructed from a flexible plastic such as, but not limited to, polyethylene terephthalate. A flexible OLED offers particular advantages of being bendable and lightweight. The invention also contemplates use of an inorganic LED. Preferably, the inorganic LED is a flexible LED. The present disclosure contemplates preferred embodiments a plurality of LEDs comprising at least one inorganic LED and at least one organic LED. Other embodiments relating to an LED contemplate a quantum dot LED.

Various laser or non-coherent light sources are suitable for use in the present invention including inert gas lasers and semiconductor laser diodes such as helium neon (HeNe; about 633 nm), ruby (about 694 nm), argon (about 488 nm and about 514 nm), krypton (about 521, about 530, about 568, about 647 nm), gallium arsenide (GaAs; >about 760 nm, with a common example of about 904 nm), and gallium aluminium arsenide (GaAlAs; about 612 to about 870 nm). Ranges are to be interpreted as being fully inclusive of all values between the limits.

Delivery of the electromagnetic radiation and in particular delivery of an electromagnetic radiation by an LED device may be either continuous or in a pulsed mode with specific pulse sequences and durations. The choice of either continuous mode or pulsed mode is dependent on the application as is known by a skilled addressee. It is also contemplated that certain embodiments employ a combination of a continuous mode and a pulsed mode.

Any suitable power source may be included in the device of the present disclosure. The power source may be a full battery unit integrated into the electromagnetic radiation source. Alternatively, the power source may be configured to be rechargeable, for example by inclusion of an AC power socket or a USB socket. Also contemplated are renewable energy sources such as solar power. It will be appreciated that the power source may be configured to suit a particular electromagnetic radiation source, as will be known by a person of skill in the art.

Preferred embodiments relating to devices of the present disclosure include a timing circuit configured to stop the electromagnetic radiation after a predetermined amount of elapsed time. Such a timing circuit is particularly advantageous for a pulsed mode of operation. For example, a predetermined amount of elapsed period of time may be about 10 seconds, about 20 seconds, about 30 seconds, about 40 seconds, about 50 seconds, and about 60 seconds or more, 1 hour or 2 hours. The predetermined amount of elapsed time is dependent on the application.

The present disclosure contemplates embodiments that include an adjustor to adjust a wavelength of electromagnetic radiation emitted from the electromagnetic radiation source. This is advantageous if a plurality of wavelengths is desired. By way of example, the device may include, or be operably connected to a red LED device configured to emit light at any wavelength in the red spectral range and an adjustor for adjusting to a wavelength between about 630 nm and about 700 nm accordingly. A conventional adjustor is contemplated and may include a processor.

Devices of the present disclosure may include a monitoring assembly. The monitoring assembly may include a controller connected to sensors for sensing various aspects of a subject while in treatment. It will be appreciated by those skilled in the art that a wide variety of properties can be sensed by a large number of types of sensors such as, but not limited to, heart rate, quantity of radiation, temperature etc. The sensors may be connected to a controller by wires, although in alternative embodiments, wireless transmission is also envisaged. The controller may be connected to a transceiver that is configured for being connected electronically to either a mobile computing device such as a smart phone or the like, or a computing device such as a desktop personal computer or to cloud-based servers (not shown) over the Internet. The transceiver can be configured for being connected by a wired connection, for example by including connectable ports for connection by adapters, or including a cord and plug for connecting to other electronic devices.

Alternately, a transceiver can be configured to connect wirelessly to outside electronic devices. The transceiver may utilise any one or more of several wireless protocols and/or network types, including wireless wide area networks (WWAN), wireless local area networks (WLAN), wireless personal area networks (WPAN) and/or wireless sensor actor networks (WSAN). In a most preferred embodiment, the transceiver will be configured for receiving and/or transmitting wireless signals over Bluetooth and/or IEEE 802.11a/b/g/n/ac (“Wi-Fi”) and/or near field communication (NFC). In an alternative embodiment, it is envisaged that a transceiver need not be provided, and instead a transmitter need only be provided, that receives signals from the sensors and transmits them on widely. It will also be appreciated that in an alternative embodiment a controller need not necessarily be provided, as the controlling functionality can be provided by a remote terminal such as a mobile phone or desktop. In a preferred embodiment, a mobile phone and/or computing device will preferably run through a downloadable application or mobile application (“app”) that is downloadable from any one of a number of available mobile application virtual stores such as Google Play or Apple App Store. Also contemplated is use of “smartphone” technology to send information to outside devices such as an LCD screen, a timer, or an app, although without limitation thereto.

The device of the present disclosure may include a manual input interface configured for receiving input from a user. Non-limiting examples of said input include frequency, intensity, wavelength and duration of treatment.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the present disclosure without limiting the present disclosure to any one embodiment or specific collection of features. Those of skill in the art will therefore appreciate that, in light of the instant disclosure, various modifications and changes can be made in the particular embodiments exemplified without departing from the scope of the present disclosure. All such modifications and changes are intended to be included within the scope of the appended claims.

1-9 (canceled).

10. A device for targeted delivery of electromagnetic radiation to a subject, said device comprising:
a first optical fibre capable of transmitting electromagnetic radiation through blood to a site in need thereof, or is capable of transmitting electromagnetic radiation to a cell, a surface, a tissue and/or an organ of the subject; and
a second optical fibre capable of receiving light.

11. The device of claim 10, wherein the second optical fibre further includes a camera.

12. The device of claim 10, which further includes an electromagnetic radiation source.

13. The device of claim 12, wherein the electromagnetic radiation source is configured to emit light.

14. The device of claim 10, which further includes a securing member configured to secure the device to at least a portion of the subject.

15. The device of claim 14, wherein the securing member includes strapping.

16. The device of claim 10, which further includes an adjustor to adjust a wavelength of electromagnetic radiation emitted from the electromagnetic radiation source.

17. The device of claim 10, which further includes a manual input interface configured for receiving input from a user.

18-19. (canceled).

20. A method for activating a photosensitive drug in a remote location in a subject, the method including:
delivering the photosensitive drug to a target area in need of treatment; moving a fibre optic light source into contact with a blood stream in fluid communication with the target treatment area; and
delivering light to the target treatment area to activate the photosensitive drug, the delivering of the light including transmitting the light from the fibre optic source into the blood stream along a non-linear path to the target treatment area.

21. The method of claim 20, wherein the fibre optic light source is configured to transmit non-visible light.

22. The method of claim 20, further comprising injecting a blood-thinning drug into the blood stream prior to delivering the light to the target treatment area.

23. A system for intra-arterially irradiating a target treatment area within a subject, comprising:
an electromagnetic radiation source including at least one optic fibre configured for passage through a catheter, said optic fibre including a tip adapted to transmit electromagnetic radiation to a blood stream of the subject;
an adjustment means for adjusting a frequency of the electromagnetic radiation; and
a processor configured to adjust the frequency of the electromagnetic radiation based on an estimated or measured distance from said tip to the target treatment area.

24. The system of claim 23, wherein the electromagnetic radiation source is configured to emit a wavelength between about 10 nanometres and about 1 millimetre.

25. The system of claim 24, wherein the electromagnetic radiation source is configured to emit a wavelength between about 100 nanometres and about 500 micrometres.

26. The system of claim 25, wherein the electromagnetic radiation source is configured to emit a wavelength between about 400 nanometres and about 100 micrometres.

27. The system of claim 26, wherein the electromagnetic radiation source is configured to emit a wavelength between about 400 nanometres and about 10 micrometres.

28. The system of claim 24, wherein the electromagnetic radiation source is configured to emit a wavelength between about 10 nanometres and about 1200 nanometres.

29. The system of claim 28, wherein the electromagnetic radiation source is configured to emit a wavelength between about 400 nanometres and about 1000 nanometres.

30-31. (canceled).
